(19) United States
(12) Patent Application Publication
(10) Pub. No.: US 2026/0192023 A1
(43) Pub. Date: Jul. 9, 2026

(54) DEVICES AND METHODS FOR LUBRICATING SURGICAL SYSTEMS

(71) Applicant: OLYMPUS CORPORATION, Tokyo (JP)

(72) Inventors: Alex NORMAN, Harston (GB); Andrew FORBES, Harston (GB); Merissa LIM, Harston (GB); Antonia SMITH, Harston (GB); Gary STACEY, Harston (GB); Shintaro INOUE, Westborough, MA (US); Ryohei OGAWA, Hachioji-shi (JP); Thomas LEMON, Harston (GB)

(21) Appl. No.: 19/427,741

(22) Filed: Dec. 19, 2025

Related U.S. Application Data
(60) Provisional application No. 63/737,339, filed on Dec. 20, 2024.
(60) Provisional application No. 63/772,981, filed on Mar. 17, 2025.

(51) Int. Cl. A61L 29/08

(52) U.S. Cl. CPC A61L 29/08; A61L 2300/452

(57) ABSTRACT

Disclosed herein are systems, devices, and methods for lubricating and/or cleaning a medical device. Such a lubrication device may include a first portion configured to apply a lubricant to an outer surface of an elongate body of a medical device and a second portion distal to the first portion where the second portion may include a seal configured to remove the lubricant from the elongate body and a waste collection chamber configured to receive and contain the removed lubricant.

### INCORPORATION BY REFERENCE

This application claims priority to U.S. Provisional Application No. 63/737,339 filed on Dec. 20, 2024, and U.S. Provisional Application No. 63/772,981 filed on Mar. 17, 2025, each of which is hereby incorporated by reference in its entirety.

### TECHNICAL FIELD

This invention relates generally to systems, devices, and methods for lubricating and/or cleaning medical devices.

### BACKGROUND

Lubricant is often applied to medical devices prior to insertion into a patient to improve patient comfort and reduce friction. For example, during a colonoscopy, an endoscope is typically lubricated before being advanced through the rectum and colon to minimize discomfort and tissue trauma. Lubricant is usually sprayed or wiped onto the medical device manually by an operator. However, manual application of lubricant may be inefficient and inconsistent. Excess lubricant is often applied, leading to waste and unintentional lubrication of the surrounding environment or equipment. Manual application can also result in lubricant getting on the operator's gloves or hands, making it more difficult to perform the procedure or requiring the operator to stop and clean themselves. Additionally, manually applying lubricant may not achieve uniform coverage, which can compromise device performance and patient comfort. Furthermore, when the lubricated medical device is removed from the patient, residual lubricant, bodily fluids, and/or other contaminants must be manually cleaned from the device. Removing residual lubricant and other debris is especially important when the device interacts with other components of a surgical system before reprocessing (e.g., cleaning and/or sterilization). Accordingly additional systems, devices, and methods for automatically lubricating and/or cleaning medical devices are desirable.

### SUMMARY

Disclosed herein are systems, devices, and methods for applying and removing lubricant from a medical device. For example, a lubrication device may comprise a first portion configured to apply lubricant to an outer surface of an elongate body of a medical device and a second portion distal to the first portion. In some variations, the second portion comprising a seal and a collection chamber. In some variations, the seal may be configured to remove lubricant from the elongate body of the medical device. In some variations, the waste collection chamber may be configured to receive and contain the removed lubricant.

In some variations, the first and second portions may comprise a first and a second lumen therethrough respectively. In some variations, the first and second lumens may be configured to receive the elongate body of the medical device. In some variations, the first portion of the lubrication device may comprise a lubricant reservoir. In some variations, the reservoir may concentrically surround the first lumen. In some variations, the lubricant reservoir may be pressurized by one or more biasing members. In some variations, the lubricant reservoir may be pressurized by a pressurized gas. In some variations, the first portion may further comprise a plurality of lubricant transfer ports fluidly coupling the reservoir and the first lumen.

In some variations, the seal may be configured to contact the outer surface of the elongate body of the medical device received within the second lumen. In some variations, the seal may be configured to distribute the lubricant applied by the first portion when the medical device is advanced through the lubrication device in a distal direction. In some variations, the seal may be configured to remove lubricant from the outer surface of the elongate body of the medical device when the medical device is retracted through the lubrication device in a proximal direction. In some variations, the seal may be configured to direct removed lubricant into the waste collection chamber. In some variations, the seal is biased radially inward.

In some variations, the second portion may be releasably coupled to the first portion. In some variations, the first portion may comprise a lubricant inlet to receive lubricant from an external source. In some variations, the second portion comprises a lubricant outlet. In some variations, the lubricant outlet may be coupled to a negative pressure source.

Also described herein are methods for lubricating a medical device. For example, a method of lubrication a medical device may include applying lubricant to the medical device as the medical device is advanced through a lumen of a lubrication device, removing lubricant from the elongate body of the medical device as the medical device is retracted through the lubrication device, and capturing the removed lubricant within the lubrication device. In some variations, the removed lubricant may be captured in a waste collection chamber of the lubrication device.

Also described herein are lubrication devices. A lubrication device may comprise a first portion configured to apply lubricant to an endoscope when the endoscope is advanced through the lubrication device and a second portion distal to the first portion, wherein the second portion is configured to remove the lubricant from the endoscope when the endoscope is retracted through the lubrication device.

Also described herein are methods for lubricating an endoscope. A method of lubricating an endoscope may include applying lubricant to the endoscope as the endoscope is advanced through a lubrication device and into a patient, and removing lubricant from the endoscope as the endoscope is retracted from the patient through the lubrication device.

Also described herein are surgical systems. A surgical system may include a medical device, a drive device releasably coupled to the medical device where the drive device is configured to control the medical device, and a lubrication device slidably coupled to the medical device distal to the drive device. The lubrication device may include a first portion configured to apply a lubricant to an outer surface of the medical device and a second portion distal to the first portion where the second portion may comprise a seal configured to remove the lubricant from the medical device and a waste collection chamber configured to receive and contain the removed lubricant.

### BRIEF DESCRIPTION OF THE DRAWINGS

FIG. 1 depicts an illustrative variation of an endoscope system.

FIG. 2 depicts a perspective view of an exemplary lubrication device.

FIG. 3 depicts a cross-sectional side view of the exemplary lubrication device of FIG. 2.

FIG. 4A depicts a cross-sectional side view of an exemplary lubrication device applying lubricant to a medical device. FIG. 4B depicts a cross-sectional side view of an exemplary lubrication device removing lubricant from a medical device.

FIG. 5A depicts a cross-sectional side view of an exemplary lubrication device including a plurality of pressurized reservoirs. FIG. 5B depicts a cross-sectional side view of a pressurized reservoir of the lubrication device of FIG. 5A.

FIG. 6 depicts a cross-sectional side view of an exemplary lubrication device including a reservoir and compressible membrane.

FIG. 7 depicts a cross-sectional side view of an exemplary lubrication device including a high-pressure gas cannister.

FIG. 8 depicts a cross-sectional side view of an exemplary lubrication device including a plurality of biasing members.

FIG. 9A depicts a cross-sectional side view of an exemplary lubrication device including an activation pin. FIG. 9B depicts a top view of the exemplary lubrication device of FIG. 9A.

FIG. 10 depicts a cross-sectional side view of an exemplary lubrication device comprising a biasing member around a lumen of the device.

FIG. 11 depicts a cross-sectional side view of an exemplary lubrication device and a plug inserted therein.

FIG. 12A depicts a perspective view of a plug comprising a plurality of caps. FIG. 12B depicts a perspective view of an exemplary lubrication device with the plug of FIG. 12A inserted therein.

FIG. 13 depicts a cross-sectional side view of an exemplary lubrication device including a helical applicator.

FIG. 14 depicts a cross-sectional side view of an exemplary lubrication device including a plurality of bristles.

FIG. 15 depicts a cross-sectional perspective view of an exemplary lubrication device including a lubricant inlet and a waste outlet.

FIG. 16 depicts a cross-sectional side view of a portion of the exemplary lubrication device of FIG. 15.

FIG. 17 depicts a cross-sectional side view of an exemplary motorized lubrication device.

FIG. 18 depicts a cross-sectional front view of an exemplary motorized lubrication device.

### DETAILED DESCRIPTION

Generally, described herein are systems, devices, and methods for applying and/or removing lubricants from a medical device, such as an endoscope. Applying lubricant to a medical device may reduce the amount force required to advance the device into a patient, such as, for example, through a body lumen of the patient or through another medical device already inserted into the patient. This reduction in force may improve patient comfort and reduce the trauma to tissue, allow an operator to more easily perform a procedure, or reduce the force a surgical system provides to advance the medical device. Removing lubricant from the medical device may allow the device to be handled by an operator or surgical system without complete reprocessing or sterilization of the device. The systems, devices, and methods described herein may automatically apply lubricant as a medical device is advanced through the lubrication device and/or remove residual lubricant and other bodily fluids and materials from the medical device as it is retracted through the lubrication device. Thus, the lubrication systems, devices, and methods described herein may provide several advantages including: more evenly distributing lubricants on a medical device, reducing the amount of lubricant need to lubricate the medical device, reducing the amount of lubricant and contaminants unintentionally transferred to an operator, other devices or system components, and the surroundings, reducing the amount of residual lubricant and contaminants on the medical device when it is retracted, and reducing the amount of residual lubricant and contaminants unintentionally transferred from the medical device to an operator or surgical system.

Lubrication devices as described herein may be configured to apply and/or remove lubricant to a medical device. A lubrication device may apply lubricant to an outer surface (e.g., sheath, covering, sleeve) of the medical device and, in some variations, may remove waste from the outer surface. In some variations, the lubrication device may be configured to apply lubricant to and/or remove waste from the outer surface of an elongate body (e.g., shaft) of the medical device. As referred to herein, applying lubricant to or removing waste from a medical device may be understood to, in some variations, relate only to application to and/or removal from the outer surface of the medical device.

Generally, the lubrication device may transfer lubricant to the medical device. The lubrication device may be configured to store lubricant, for example, in one or more reservoirs. The device may transfer the lubricant from the one or more reservoirs to the medical device. The reservoir, in some variations, may be pressurized to improve the transfer of lubricant to the medical device. For example, one or more biasing members (e.g., springs), a pressurized gas, or a lubricant inlet coupled to a positive pressure source may pressurize the reservoir. In some variations, the lubrication device may comprise one or more distribution features (e.g., seals, bristles, or the like) to evenly distribute the lubricant on the medical device.

Moreover, the lubrication device may be configured to remove lubricant and/or bodily fluids or other contaminants from the medical device. For example, a seal of the lubrication device may be configured to contact the medical device to remove lubricant and/or contaminants therefrom. In this manner, the seal may wipe and/or scrape waste, including residual lubricant and/or contaminants (e.g., bodily fluids and other materials on the device after removal from the body), off the medical device. The seal may be biased towards a portion of the device configured to receive the medical device. For example, the seal may be biased towards a central lumen of the device. The seal may be further configured to direct waste removed from the medical device into a collection chamber. For example, the lubrication device may itself comprise a waste collection chamber for receiving and/or storing waste. Additionally or alternatively to a seal, the lubrication device may be configured to apply negative pressure to the medical device to remove waste therefrom. In this manner, the lubrication device may flush waste from the device.

Also described herein are methods for lubricating and/or removing lubricant from medical devices. For example, a method of lubricating a medical device may include coupling the lubrication device to the medical device. In some variations, a lubricant transfer port seal such as a plug or a cover may be removed or a reservoir storing the lubricant may be pressurized prior to applying the lubricant. The method may include applying lubricant to the medical device using a lubrication device as described herein. The lubricant may be applied as the medical device is advanced through the lubrication device. A method of lubricating a medical device may further include removing lubricant from the medical device using the lubrication device. Waste may be removed from the medical device as the medical device is retracted through the lubrication device, for instance, using one or more seals of the lubrication device. In some variations, the method may further include collecting the lubricant and/or contaminants removed from the medical device within the lubrication device. The collected waste may be properly disposed of as medical waste.

Devices and methods described herein may be used in conjunction with a surgical system. Generally, a surgical system as described herein may comprise a medical device and one or more control devices for controlling the medical device. Coupling a lubrication device as described herein to the medical device may prevent waste on the medical device from contaminating the one or more control devices.

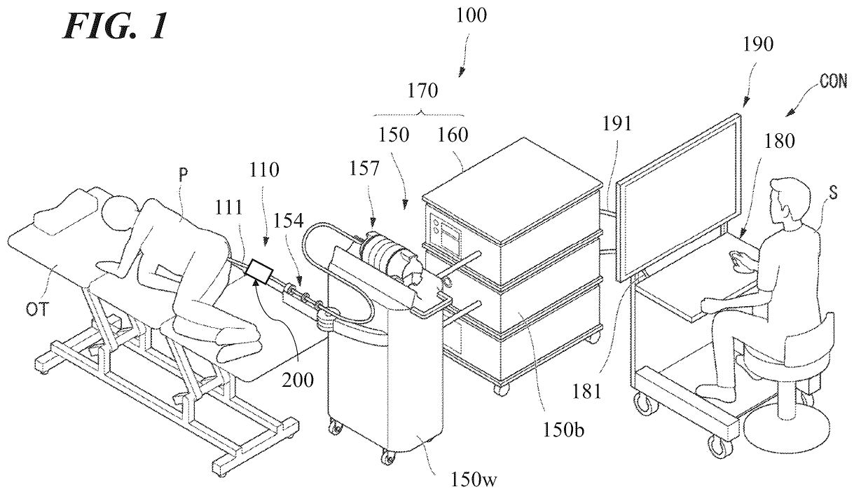

FIG. 1 shows a surgical system 100 with which the lubrication devices described herein may be used. The surgical system 100 is an example of an electronic endoscope system. The surgical system may include a medical device (e.g., insertion manipulator 110) inserted into the body (P), an electric endoscope, a catheter, a treatment tool, an endoluminal device, and the like, any of which may be lubricated or cleaned by the lubrication devices and methods described herein. The surgical system 100 may be a medical system for observing and treating the inside of the body of a patient (P) lying on an operating table (OT). The surgical system 100 may include an insertion manipulator 110, a treatment manipulator (not show), a lubrication device 200, a drive device 150, an image control device 160, an operation device 180, and a display device 190. The drive device 150 and the image control device 160 may constitute a control device 170 that controls the surgical system 100. A lubrication device, as described herein, may be coupled to insertion manipulator 110 such that upon reaction of the insertion manipulator, the insertion manipulator is cleaned before reaching the control device 170.

The insertion manipulator 110 may be a device inserted into a lumen of a patient to observe and treat a lesion. The insertion manipulator 110 may have excellent insertability and can be inserted into the gastrointestinal tract of the patient, for example, an ascending colon or a cecum of a large intestine. The lubrication device 200 may be slidably coupled to the insertion manipulator 110. In some variations, an elongate body of the insertion manipulator 110 may be inserted through the lubrication device 200 proximal to the patient (P). The insertion manipulator 110 may be detachably attached to the drive device 150 and the video control device 160. An internal path may be formed in the insertion manipulator 110.

The treatment manipulator (not shown) may be, for example, a device inserted into a first channel tube of the insertion manipulator 110, protrudes from a first opening, and is inserted into a lumen of a patient to treat a lesion. An end effector (a treatment portion) that treats a lesion may be disposed at the distal end of the treatment manipulator. The drive device 150 may be detachably connected to the insertion manipulator 110 and the operation device 180. The drive device 150 may be configured to control or at least partially control the medical device 110 (e.g., insertion manipulator). The drive device 150 may drive a motor built thereinto on the basis of an operation input to the operation device 180 to electrically drive the insertion manipulator 110. The drive device 150 may drive a pump or the like built thereinto on the basis of an operation input to the operation device 180 and causes the insertion manipulator 110 to perform air supply, water supply, and suction. The drive device 150 may include a main drive unit 157 and an insertion drive unit 154. The insertion drive unit 154 may assist with inserting the insertion manipulator 110 into the body of the patient (P). The insertion drive unit 154 may be mounted on an arm that extends deformably from the cart 150W. The video control device 160 may be detachably connected to the insertion manipulator 110 and acquires a captured image from the insertion manipulator 110. The video control device 160 may cause the display device 190 to display the captured image acquired from the insertion manipulator 110 or a GUI image or a CG image to provide information to an operator.

The drive device 150 and the video control device 160 may constitute a control device 170 that controls the electric endoscope system 100. The control device 170 may further include peripherals such as a video printer. The drive device 150 and the video control device 160 may be a unified device. The operation device 180 may be detachably connected to the drive device 150 via an operation cable 181. The operation device 180 may communicate with the drive device 150 in a wireless manner instead of in a wired manner. An operator(S) may electrically drive the insertion manipulator 110 by operating the operation device 180. The display device 190 may be a device that can display an image such as an LCD. The display device 190 may be connected to the video control device 160 via a display cable 191. The operation device 180 and the display device 190 may be mounted in a trolley or console.

Generally, the lubrication device 200 may be positioned along the length of the medical device 110. In some variations, the medical device 110 may be inserted through a lumen of the lubrication device 200. The lubrication device 200 may be releasably coupled to the medical device 110 to apply lubricant. The lubrication device 200 may apply lubrication prior to the medical device 110 entering the patient (P). The lubrication device 200 may be coupled to the medical device 110 distal to the drive device 150 and/or control device 170 to clean (i.e., remove waste) from a contaminated portion the medical device before that portion is handled by the drive unit or control device. The lubrication device 200 may be distal to insertion drive unit 154 to prevent contamination of the insertion drive unit and other proximal components. Alternatively, the lubrication device 200 may coupled to the medical device 110 proximal to the insertion drive unit 154 but distal to the main drive unit 157. In some variations, the lubrication device 200 may be coupled to the arm of the cart 150w. The lubrication device may coupled (e.g., releasably coupled) to the insertion drive unit 154 using one or more fasteners. The lubrication device may remove waste from the medical device when the medical device is retracted from the patient (P) by the drive unit 115.

The devices and methods described herein may be used with one or more water-based lubricants, silicone-based lubricants, oil-based lubricants, or other lubricant suitable for surgical operations. The devices and methods may use lubricants that are biologically inert. The lubricant may have a viscosity of about 1000 mPa·s to about 9000 mPa·s such that the lubricant may reduce the fictional force and dynamic coefficient of friction between the medical device and the patient, another device, and/or another system component. The lubricant may reduce the dynamic coefficient of friction between the outer surface of the medical device (e.g., sheath) may be about 0.15 to about 0.08.

### I. Devices

Generally, the devices described herein may apply lubricant to a medical device and remove waste (e.g., residual lubricant, contaminates) from the medical device after the medical device has been in the patient. Lubrication devices described herein may be configured to lubricate a variety of medical devices including endoscopes, laparoscopes, catheters, trocars, implants, or the like. The devices described herein may be used during a medical procedure in which lubricating a medical device used in the procedure would increase patient comfort such as a colonoscopy, gastroscopy, rectoscopy, bronchoscopy, cystoscopy, catheterization procedure, or the like. The devices described herein may also be used during a medical procedure in which an operator or surgical system may normally handle a contaminated medical device, including any of the aforementioned procedures. In some variations, the lubrication device or a portion thereof may be single-use. For example, a portion of the device used to collect waste (e.g., collection chamber) may be disposed of after a single patient's procedure or the entirety of the device may be disposed of after a single patient's procedure.

In some variations, the lubrication device may be configured to fit around the medical device (e.g., an endoscope) to apply lubricant. The lubrication device may be elongated and may cover a length of the medical device. In some variations, the lubrication device may be slidably coupled to the medical device but may remain fixed relative to movement of the medical device (i.e., such that the medical device may translate relative to the lubrication device). The lubrication device may have a lumen extending therethrough the configured receive a medical device, such as an endoscope. The lumen may extend longitudinally though the lubrication device from a distal end to a proximal end. A dimension of the lumen may be about the same size as an outer dimension of the medical device to control the thickness of a layer of lubricant applied. In some variations, the lubrication device may have a circular profile. The lubrication device may have a cylindrical shape.

The lubrication device may comprise a first portion for applying lubricant and a second section for removing lubricant. The first portion may be configured to store and transfer lubricant onto the medical device. In some variations, the first portion may apply the lubricant when the medical device is advanced through the lubrication device (e.g., towards the patient). The first portion of the lubrication device may use positive pressure applied externally or generated within the first portion to assist in application of the lubricant. The first portion may distribute lubricant to a plurality of points about a circumference of the medical device to more evenly distribute the lubricant. Additionally or alternatively, the first portion may apply lubricant to all or nearly all of the circumference of the medical device. In some variations, the first portion may be configured to store the lubricant securely before application.

The second portion of the lubrication device may be configured to remove lubricant and/or contaminants (e.g., bodily fluids, particulates, and/or other waste) from the medical device. In some variations, the second portion may be distal to the first portion such that that the medical device may be advanced through the first portion before reaching the second portion. However, the second portion may not necessarily be positioned distal to the first portion so long as the second portion is positioned to remove residual lubricant and/or contaminants before the medical device is handled by an operator or surgical system. In some variations, one or more separate portions may remove waste from the medical device. For example, a second portion of the lubrication device may remove larger solid waste from the medical device, and a third portion of the lubrication device may remove smaller or liquid waste from the medical device. In some variations, the second portion may collect and store the waste removed from the medical device.

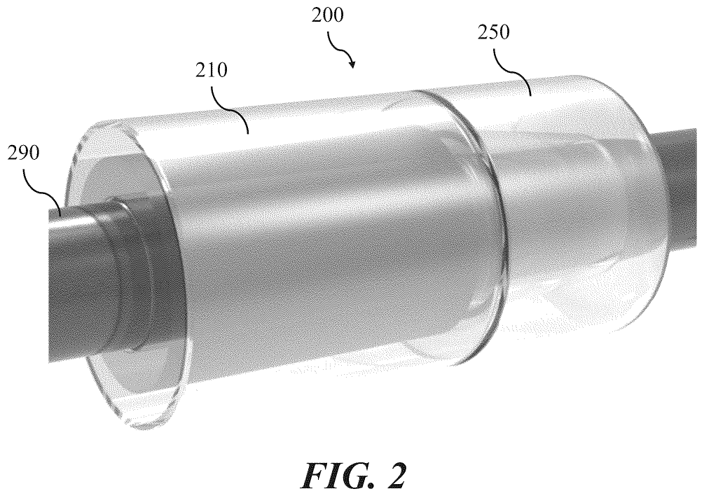

FIG. 2 depicts a perspective view of an exemplary lubrication device 200 slidably coupled to a medical device 290. The lubrication device 200 may comprise a first portion 210 configured to apply lubrication to the medical device 290 and a second portion 250 configured to remove waste. The second portion 250 may be distal to the first portion 210. In some variations, the first and second portions may be releasably coupled to each other. In other variations, the first and second portions may be formed integrally as part of the same device. In some variations, as shown in FIG. 2, one or more portions of the lubrication device 200 may be transparent. A transparent first portion of the lubrication device may allow an operator to monitor a volume of lubricant stored in the first portion 210. A transparent second portion 250 may allow an operator to monitor removal and/or or collection of waste from the medical device.

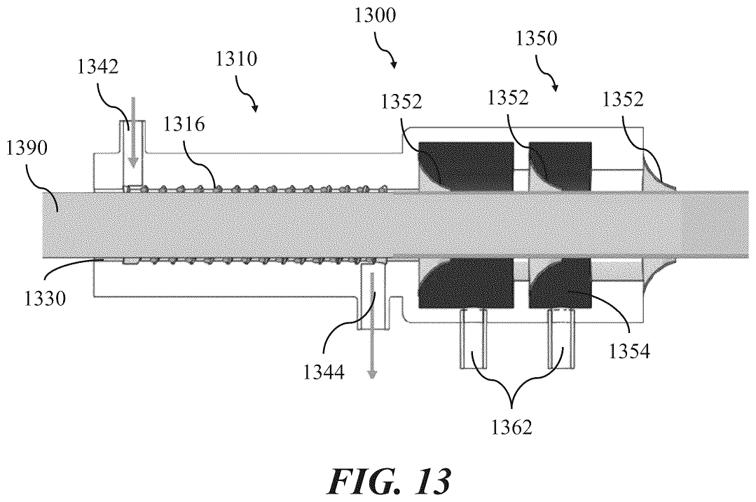

In some variations, the first and second portions may be releasably coupled to one another. This may be advantageous when one portion of the lubrication device is single-use and another portion is reusable. For example, the second portion of the lubrication device may be configured to be detached from the first portion of the device and disposed of after it has removed and collected waste. The first portion and the second portion may be releasably coupled to one another by a fastener, such as one or more of a latch, a screw, or a clasp. In some variations, the lubrication device may comprise an actuator to release the second portion from the first portion (See FIG. 14).

In some variations, the first portion may be configured to store lubricant. The first portion may comprise one or more lubricant reservoirs to store lubricant. In some variations, the first portion may store lubricant around a lumen of the first portion. In this configuration, the first portion may be configured to flow lubricant radially inward (e.g., towards a lumen through the first portion). In this manner, the first portion may transfer lubricant to the medical device. The lubricant may be transferred from the lubricant reservoir to the medical device. As mentioned previously, the first portion of the lubrication device may comprise a lumen configured to receive the medical device for lubrication. The diameter of the lumen may be about equal to an outer diameter of the medical device (e.g., an outer diameter of a shaft of the medical device). A close fit between the lumen and the medical device may more evenly apply the lubricant. The first portion may be preloaded with lubricant, have an inlet for loading lubricant, or have an inlet and outlet for flowing lubricant through the lubrication device. For example, a lubricant reservoir of the first portion of the lubrication device may be pre-filled with lubricant. In some variations, a pressure source (e.g., a high-pressure source such as compressed air) may be coupled to the inlet to pressurize the lubricant. Pressurizing the lubricant may force the lubricant through one or more ports (e.g., lubricant transfer ports) and onto the medical device.

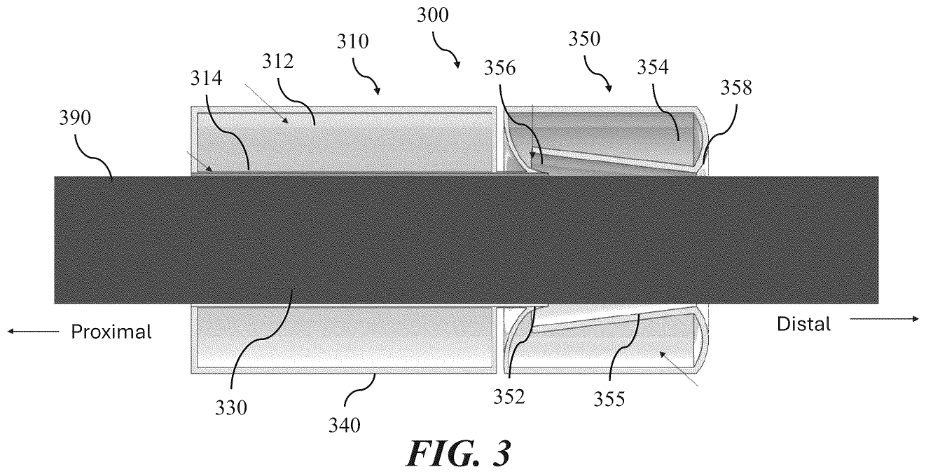

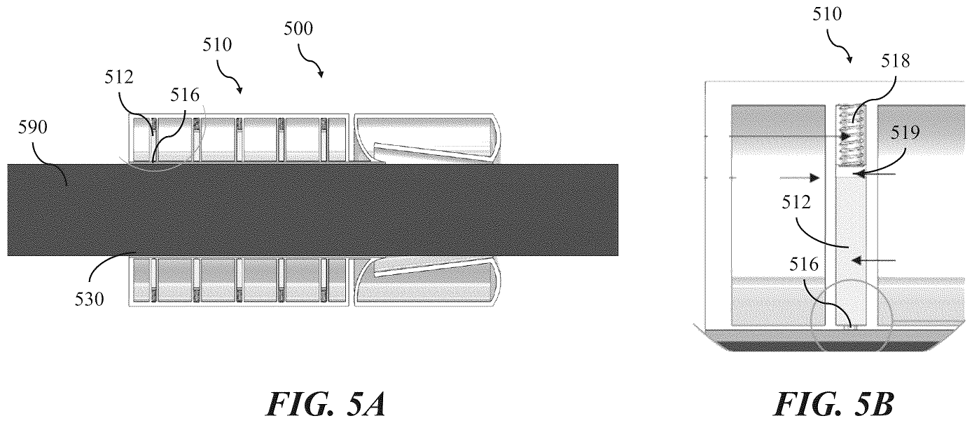

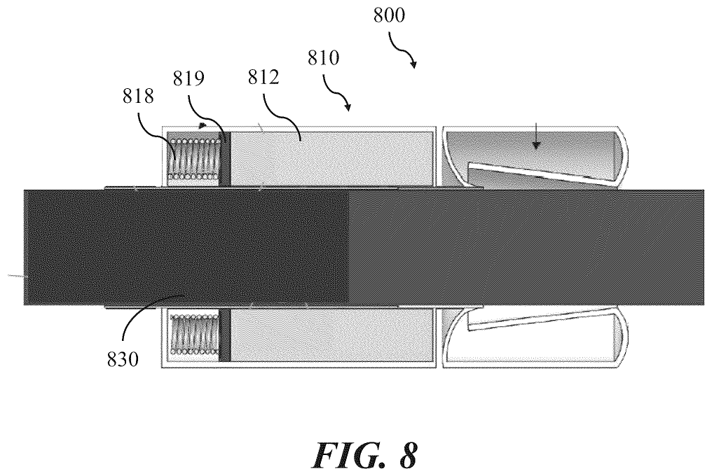

FIG. 3 depicts a cross-sectional side view of an exemplary lubrication device 300 including a first portion 310 and a second portion 350 The first portion may comprise a housing 340. In some variations, the first portion 310 and the second portion 350 are formed integrally as part of the same housing 340. In some variations, the housing 340 may define one or more reservoirs, chambers, or cavities of the lubrication device. For example, the first portion may comprise a lubricant reservoir 312. The lubricant reservoir 312 may be within the housing 340. In some variations, the lubricant reservoir 312 may be pressurized such that a pressure is applied to the lubricant. The lubricant reservoir 312 may be pressurized by one or more biasing members, a pressurized gas, or a membrane as will be discussed in more detail herein. In some variations, a longitudinal axis of a biasing member may be parallel to the longitudinal axis of the lubrication device (e.g., biasing member 818 of FIG. 8). In other variations, the longitudinal axis of the biasing member may be perpendicular to the longitudinal axis of the lubrication device (e.g., biasing member 518 of FIG. 5B). The first portion 310 may comprise an inner wall 314 separating the lubricant reservoir 312 from the lumen 330.

In some variations, the first portion 310 may have one or more lubricant transfer ports to transfer lubricant from the lubricant reservoir 312 to the medical device 390. The lubrication device 300 may comprise 1, 2, 3, 4, 5, 6, 8, 10, 12, 16 20, 24, 30, or more than 30 lubricant transfer ports. The lubricant transfer ports may each comprise an opening between the lubricant reservoir 312 and the lumen 330. The openings may be sized such that there is no, or insignificant lubricant flow naturally through the openings due to the viscosity of the lubricant without an additional force or pressure being applied to the lubricant. In some variations, in addition to or as an alternative to the lubricant transfer ports, the first portion may comprise a helical applicator and/or bristles for applying lubrication to the medical device. In some variations, the lubricant transfer ports may be plugged prior to application of the lubricant (e.g., during storage and/or transit).

In some variations, the second portion may be configured to remove and store waste. The second portion may, in some variations, mechanically wipe, scrape, and/or squeeze waste off the medical device. The second portion may comprise one or more seals to remove the waste. The seal may comprise an elastomer such as silicone, silicone rubber, neoprene, or other flexible material. The second portion, and particularly the seal, may have a lumen configured to receive a medical device. The lumen of the second portion (e.g., second lumen) may be coaxially aligned with a lumen of the first portion (e.g., first lumen) to form a lumen of the lubrication device. In some variations, a diameter of the seal lumen may be about the same as a diameter of the medical device. This may allow the seal to remove waste on the outer surface of the medical device as the medical device is moved relative to the seal. In some variations, the seal may be biased radially inward. For example, the seal may be biased radially inward toward the lumen or medical device inserted therein. In some variations, the seal may deflect radially outward when a medical device is advanced through the lubrication device. The seal may distribute the lubricant applied by the first portion to more evenly coat the medical device. The seal may deflect radially inward to closely fit an outer surface of the medical device when the medical device is retracted through the lubrication device. In this manner, the seal may remove waste from the medical device.

In some variations, the second portion may receive and/or store the removed lubricant and other bodily waste therein. Collection of waste may avoid contaminating an operator or system handling the lubricated medical device when removed from the patient. In some variations, the second portion may comprise a waste collection chamber. The waste collection chamber may be positioned relative to the seal such that waste scraped off the medical device is directed into the waste collection chamber. In some variations, the waste collection chamber may be disposed of after using the lubrication device.

Returning to FIG. 3, The second portion 350 of the lubrication device 300 may comprise one or more seals 352 (e.g., two, three, four, or more) and a waste collection chamber 354. The seal 352 may be positioned proximally within the second portion 350 and may be distal to the first portion 310 to remove waste before it reaches the first portion 310. In some variations, the seal 352 may be annular. The seal 352 may be configured to contact the circumference of the medical device when the medical device 390 is within the seal 352. For example, the seal 352 may be coupled to internal surface of the lubrication device 300 and extend radially inward towards the lumen 330. The seal 352 may extend inward such that an end of the seal contacts an outer surface of the medical device 390. In some variations, the seal 352 may extend into the lumen 330 in its natural undeformed state. The seal 352 may be biased radially inward. The seal 352 be configured to deform radially outward and deform such that the innermost part of the seal deflects in a distal direction. In some variations, the seal 352 may comprise a bend configured to direct waste into the waste collection chamber 352. The second portion 350 may comprise a channel 356 formed at least partially by the bend in the seal through which waste may be directed into the waste collection chamber 354.

In some variations, the waste collection chamber 354 may receive and contain waste removed by the seal 352. The waste collection chamber 354 may take up a majority of the volume of the second portion 350. The waste collection chamber 352 may be annular and may surround the lumen 330. The second portion 350 may comprise an inner wall 355 to separate the waste collection chamber 354 and the lumen 330. In some variations, an inner diameter of the inner wall 355 may be greater than the inner diameter of the seal 352 (i.e., diameter of the seal lumen). This may prevent waste from sticking to the inner wall 355 and instead direct waste to the seal 352, which may direct the waste to the waste collection chamber 354 to receive the waste. In some variations, the inner diameter of the inner wall may increase in the proximal direction. A distal end of the second portion 358 may be flared to direct waste into the second portion 350. In some variations, the second portion 350 may comprise an outlet to remove waste from the second portion 350. The outlet may be coupled to a negative pressure source to suction waste from the waste collection chamber 354.

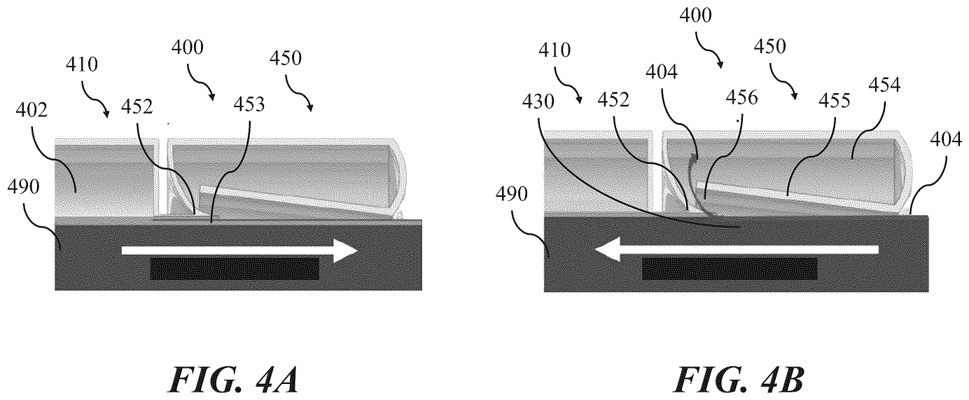

In some variations, the seal may more evenly distribute lubricant on the medical device when it is advanced through the lubrication device. FIG. 4A shows a lubrication device 400 with a medical device 490 advancing in the direction shown. Generally, a first portion 410 of the lubrication device may be configured to apply lubricant as described herein. A seal 452 of a second portion of the lubrication device 450 may be configured to more evenly distribute the lubricant 402 applied by the first portion 410. For example, when the medical device 490 is advanced in the direction shown (i.e., distally) (e.g., towards the patient) the seal 452 may more evenly distribute the lubricant 402. The seal 402 may be configured to adjust the thickness of a layer of lubricant 402 applied to the medical device 490. In some variations, the seal 452 may be configured to deflect radially outward as the medical device 490 is advanced in the direction shown. In this manner, the seal 452 may be configured to apply lighter pressure (i.e., a lesser force) to the medical device when the medical device 490 is advanced compared to when the medical device 490 is retracted in an opposite direction. In some variations, the seal 452 may be configured to deflect to open a gap 453 between the seal 452 and the medical device 490 to control distribution of the lubricant. For example, the seal may deflect radially outward to allow a coat of lubricant with a thickness about the height of the gap 453 to be applied to the medical device. In some variation a gap formed by a seal or another component of the lubrication device configured to distribute lubricant may be about 0.1 mm to about 1 mm, such as about 0.5 mm, including all subranges and values therebetween.

FIG. 4B shows a lubrication device 400 with a medical device 490 retracting in the direction shown. The second portion of the device 450 may be configured to remove and/or collect waste 404 (e.g., residual lubricant, bodily fluids) from the medical device 490 as it is retracted in the direction shown (i.e., distally) (e.g., away from the patient). In some variations, the seal 452 may be configured to deflect radially inward to remove waste 404. In this manner, the seal 452 may apply an increased pressure (i.e., force) to the medical device 490 as the medical device is retracted compared to the pressure applied while the medical device advanced. This increase in contact pressure may allow the seal 452 to scrape or wipe waste 404 from the medical device 490. In some variations, the seal 452 may be configured to decrease and/or close a gap 453 between the seal 452 and the medical device 490. In some variations, the seal 452 may function similar to a check valve (e.g., duck valve) to allow lubricant 402 on the medical device to pass through the seal in a first direction and prevent lubricant and/or waste 404 from passing through the seal in a second, opposite direction.

In some variations, a bend of the seal 452 may direct residual lubricant and/or bodily fluids 404 into the waste collection chamber 454. For example, as shown in FIG. 4B, the seal 452 may direct waste 404 radially outward toward the waste collection chamber 454. The seal 452 may be configured to change a direction of flow of the waste 404. For example, the flow of the waste 404 may be reversed to direct the waste 404 to the waste collection chamber 454. The waste 404 may follow a bend of the seal 452 and may ultimately flow in a longitudinal direction opposite the direction of retraction of the medical device 490. In some variations, the seal 452 may be configured to direct waste 404 through a channel 456 between the lumen 430 and the waste collection chamber 454, as shown in FIG. 4B. In some variations, the waste collection chamber 454 may prevent waste 404 from flowing back to the medical device 490. For example, the inner wall 455 of the second portion 450 may be angled relative to the lumen 430 and/or medical device 490 therein to prevent waste 404 flowing out of the waste collection chamber 454.

In some variations, the first portion may include one or more pressurized lubricant reservoirs to apply lubricant to the medical device. FIG. 5A shows a cross-sectional view of an exemplary variation of a lubrication device 500 utilized pressurized lubricant reservoirs. The first portion 510 of the lubrication device 500 may comprise a plurality of lubricant reservoirs 512, as shown in FIG. 5. For example, a first portion of a lubrication device 510 may comprise 1, 2, 3, 4, 5, 6, 8, 10, 12, 16, 20, 24, 30 or more than 30 lubricant reservoirs. The number of lubricant reservoirs may be equal to the number of lubricant transfer ports. In such variations, the first portion 510 may comprise a plurality of lubrication transfer ports 516 and each lubricant reservoir 512 may correspond to a respective lubricant transfer port 516 configured to allow lubricant to flow from that lubricant reservoir 512 into the lumen 530 of the lubrication device and on to a medical device 590 therein. In some variations, each lubricant reservoir 512 may be pre-filled with lubricant prior to use.

In some variations, each lubricant reservoir may be individually pressurized. For example, FIG. 5B shows a close-up cross-sectional view of a lubricant reservoir 512 of the lubrication device 500 of FIG. 5A. As shown in FIG. 5B, the first portion 510 may comprise one or more biasing members 518 configured to pressurize the one or more lubricant reservoirs 512. In variations including a plurality of lubricant reservoirs, each lubricant reservoir may be pressurized by a respective biasing member. The biasing member may be or may comprise a spring, such as, for example, a compression spring (as shown), a torsion spring, a leaf spring, a wave spring, or the like. In some variations, the biasing member 518 may be pre-compressed such that a predetermined pressure is applied to the lubricant prior to application. The first portion 510 may comprise one or more blocks 519 (e.g., cylinder block) positioned within a lubricant reservoir 512 and. configured to seal an end (e.g., radial most end) of that lubricant reservoir. The block 519 may be slidably disposed within the lubricant reservoir 512 such that applying a force or pressure to the block expels pressurizes the lubricant in the lubricant reservoir. In some variations, the block 519 may be positioned between the lubricant and the biasing member 518. The biasing member 518 may be configured to press on the block 519 to pressurize the lubricant within the lubricant reservoir.

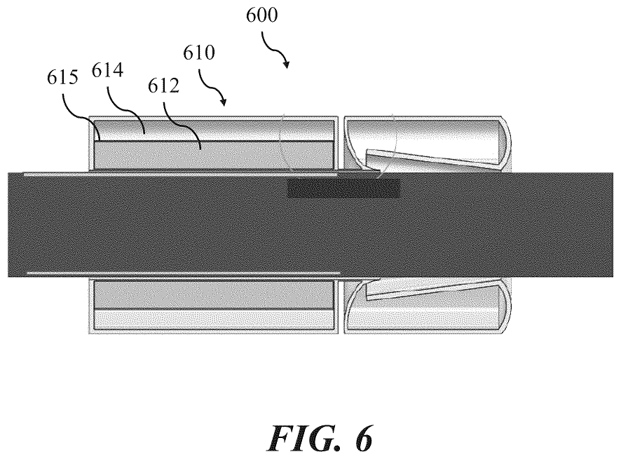

In some variations, the first portion may comprise a single lubricant reservoir. The single lubricant reservoir may be fluidly coupled to a plurality of fluid transfer ports. In some variations, the lubricant reservoir may be pressurized by a gas. The first portion may comprise a cavity containing a pressurized gas therein. The cavity may be adjacent to the lubricant reservoir such that the pressurized gas in the cavity pushes on and pressurizes the lubricant in the lubricant reservoir. FIG. 6 shows a cross-sectional view of a lubrication device 600 comprising a cavity 614 for containing a pressurized gas. As shown in FIG. 6, the first portion of the lubrication device 610 may comprise a lubricant reservoir 612, a pressurized gas cavity 614, and a membrane 615 (e.g., compressible bag) separating the cavity from the lubricant reservoir. In some variations, the cavity 614 may surround (e.g., concentrically surround) the lubricant reservoir 612 to evenly apply pressure to lubricant within the lubricant reservoir 612. The membrane 615 may be configured to deform radially inward under the pressure of the gas contained in the cavity 614. For example, the membrane 615 may deform radially inward to pressurize the lubricant in the lubricant reservoir 612 and force the lubricant through one or more transfer ports. In some variations, the cavity 614 may be pre-pressurized (e.g., provided with a predetermined pressure in the cavity) such that when a cover or plug, as described herein, is removed from the lubrication device 600, the pressurized cavity may deform the membrane 615 and dispense lubricant.

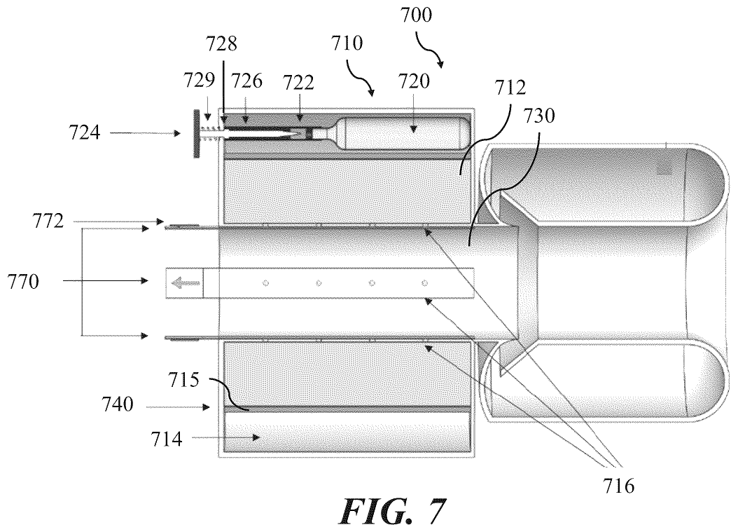

In some variations, the cavity may not be pre-pressurized and first portion may instead comprise a gas chamber (e.g., compressed CO₂ chamber) within the cavity. FIG. 7 shows a cross-sectional view of an exemplary lubrication device 700. The first portion 710 of the lubrication device may comprise a lubricant reservoir 712, a cavity 714, a membrane 715 separating the lubricant reservoir 712 from the cavity 714, and a gas chamber (e.g., a high-pressure gas chamber) in the cavity 720. The pressurized gas chamber 720 may be configured to release the gas into the cavity 714 to pressurize the cavity. The pressurized gas chamber 720 may comprise a vent 722 through which the gas is released. The first portion of the lubrication device 710 may comprise an actuator (e.g., button) 724 to release gas from the pressurized gas chamber 720 when actuated by an operator. In some variations, the actuator may be on a proximal side of the housing 740 opposite the second portion of the device. The actuator 724 may be coupled to a pin 726 that may extend though the housing 740 and pierce the pressurized gas chamber 720 when the actuator 724 is actuated (e.g., pressed). In some variations, the pin 726 may comprise an airlock 728 configured to seal the cavity 714 at the point the pin extends through the housing. The actuator 724 and the pin 726 may be biased outward from the cavity so that when the actuator is not depressed the airlock seals the cavity. For example, the first portion may comprise a biasing member 729 between the actuator 724 and the housing 740 to bias the airlock 728 to seal the cavity.

In some variations, the first portion of the device 710 may comprise one or more covers 770 to retain lubricant in the lubricant reservoir 712. A cover 770 may overlay one or more lubricant transfer ports 716 to seal the ports. In some variations, the cover 770 may be a tape. The one or more covers 770 may releasable couple to a side wall of the lumen 730. In some variations, the covers 770 may be coupled to the sidewall via an adhesive. The cover 770 may be configured to prevent a pressurized lubricant from exiting the lubricant reservoir 712. In some variations, a cover 770 may seal a plurality of lubricant ports 716. A cover may be an elongate strip. A cover 770 may comprise a tab 772 that extends outside the lumen 730 of the lubrication device. The tab 772 may be configured to be pulled by the operator to remove the cover 770. A cover may be thin enough to allow a medical device to enter the lumen while the cover is coupled to a sidewall of the lumen. In some variations, the cover may be removed from the sidewall of the lumen while a medical device is within the lumen.

In some variations, a plurality of biasing members may pressurize the lubricant reservoir. FIG. 8 shows a cross-sectional view of an exemplary lubrication device 800 comprising a plurality of biasing members 818 and a lubricant reservoir 812. As shown in FIG. 8, in some variations, the plurality of biasing members 818 may be positioned concentrically about the lumen 830. The first portion of the lubrication device 810 may comprise, for instance, two, three, four, five, six, seven, eight, or more biasing members 818 to pressurize the lubricant reservoir 812. In some variations, the plurality of biasing members 818 may be within the lubricant reservoir 812. A plunger 819 may separate the biasing members 818 from the lubricant in the lubricant reservoir 812. The plunger 819 may seal the lubricant off from the biasing member 818. In some variations, the plunger 819 may be slidably positioned within the lubricant reservoir 812. The plunger 819 may transfer force from the biasing members 818 to the lubricant to pressurize the lubricant. In some variations, the plunger 818 may be annular and may surround the lumen 830.

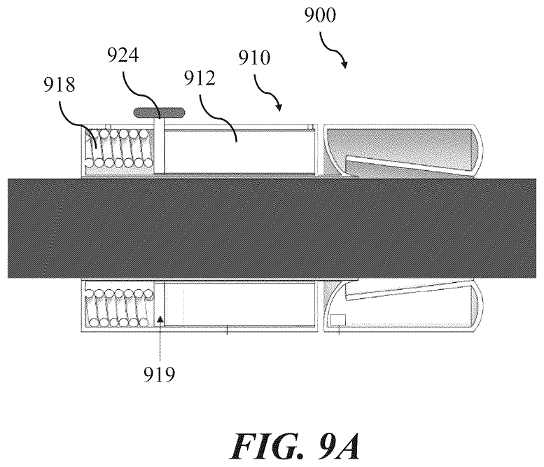

In some variations, an activation pin may prevent pressurization of the fluid prior to use. FIGS. 9A and 9B show a cross-sectional side view and a top view, respectively, of a lubrication device 900 comprising an activation pin 924. The activation pin 924 may be configured to prevent one or more biasing members 918 from pressurizing lubricant within the lubricant reservoir 912. In some variations, the activation pin 924 may prevent extension of the biasing member 918. The activation pin 924 may be coupled to a plunger 919 such that when the activation pin 924 is prevented from moving so is the plunger 919. The activation pin 924 may extend through a slot 925 in the housing 940. The slot 925 may be configured to allow the activation pin 924 to translate in a longitudinal direction and pressurize the lubricant. The slot 925 may have a notch 926 to restrain the activation pin 924. The activation pin 924 may be configured to move from the notch 926 to the slot 925 by an operator to pressurize lubricant in the lubricant reservoir 912.

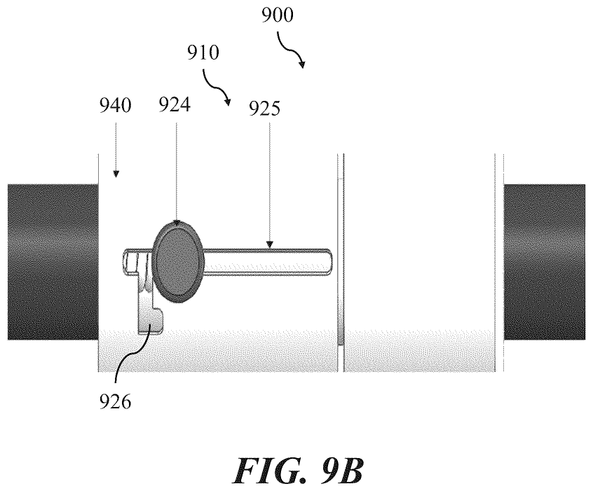

In some variations, the lubrication device may comprise a single biasing member instead of a plurality of biasing members. The single biasing member may surround a lumen of the lubrication device configured to receive the medical device. For example, FIG. 10 shows a cross-section view of the lubrication device 1000 where the first portion of the lubrication device 1010 comprises a single biasing member 1018. In some variations, a lubricant reservoir 1012 may be annular and may surround the lumen 1030. The biasing member 1018 may be within the annular lubricant reservoir 1012. In such variations, the biasing member 1018 may have a lumen such that the biasing member fits within the annular lubricant reservoir 1012. In some variations, a biasing member 1018 that surrounds the lumen 1030 of the first portion of the lubrication device 1010 may be configured to pressurize lubricant in the lubricant reservoir 1012 by applying a force to a plunger 1019. In some variations, a plunger 1019, regardless of the number or configuration of biasing members, may be configured to evenly distribute force from the biasing member to the lubricant.

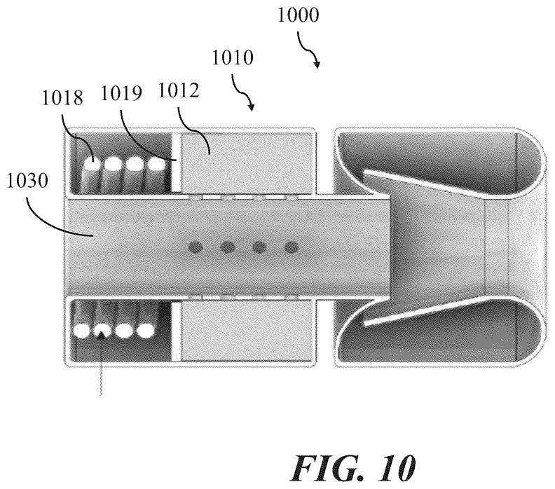

The lubrication device may include one or more components configured retain lubricant in the lubrication device (e.g., during transit, prior to application). In some variations, as mentioned herein, a lubrication device may comprise a plug to retain lubricant in the lubricant reservoir prior to application. For example, the plug may be configured to seal one or more lubricant transfer ports to prevent lubricant from flowing therethrough. FIG. 11 shows a cross-sectional view of a lubrication device 1100 including a plug 1180 inserted within the lumen 1130 of the lubrication device 1100. In some variations, the plug 1180 may be cylindrical. The plug 1180 may be longer than the first portion 1110 of the device such that a portion of the plug 1180 extends outside of the lumen 1130 of the lubrication device 1100. The plug 1180 may be slidable positioned within the lumen 1130. The plug 1180 may be configured to slide out of the lumen 1130 (e.g., in the direction show) and in doing so, uncover one or more lubricant transfer ports 1116 when removed from the lumen 1130. In some variations, the plug may be inflatable. In these variations, the plug may be configured to prevent lubricant from exiting one or more lubricant transfer ports when inflated (e.g., in-situ within the lumen) and may be configured to be removed from the lumen when deflated.

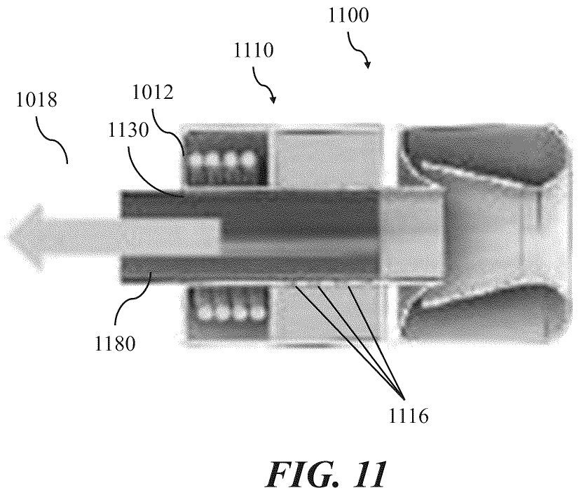

FIG. 12A shows another variation of a plug 1280 for retaining lubricant in the lubricant reservoir. FIG. 12B shows the plug 1280 inserted into a first portion of a lubrication device 1210. The plug may comprise a handle 1282 configured to unseal one or more lubricant transfer ports when pulled and a plurality of arms 1284 comprising a cap at a lateral end, where one or more caps are configured to be received within a respective transfer port of the lubrication device to block lubricant from flowing therethrough. The handle 1282 may be configured to be held by an operator such that the operator may pull on the handle 1280 to remove the plug 1280 from the lumen 1230 of the lubrication device thereby unblocking the transfer ports and allowing lubricant to flow from a lubricant reservoir. In this manner, pulling the handle 1282 may unseal one or more lubricant transfer ports. In some variations, the plurality of caps 1284 may seal lubricant within the first portion 1210. Each of the plurality of caps 1284 may correspond to a lubricant transfer port that allows lubricant to flow from the lubricant reservoir the lumen 1230. In some variations, a cap may plug (e.g., insert into) an opening of the lubricant transfer port to seal the transfer port. A plug comprising a plurality of caps may advantageously use less material compared to a cylindrical plug.

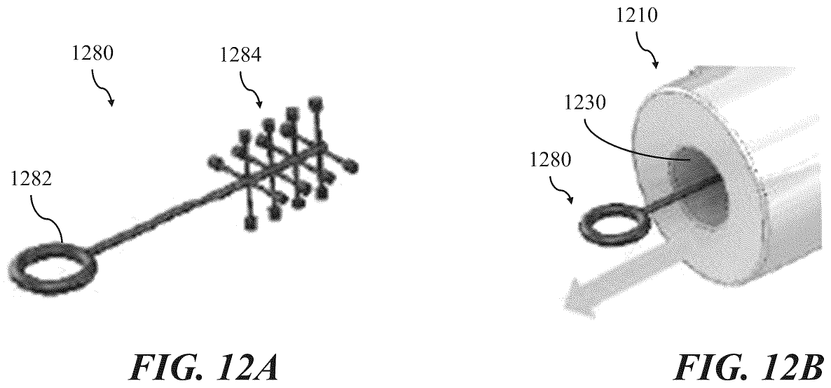

In some variations, the first portion of the lubrication device may comprise a lubricant inlet and/or a lubricant outlet. In these variations, the first portion may be configured to apply lubricant to a medical device as the lubricant flows from the lubricant inlet to the lubricant outlet. For example, FIG. 13 shows a cross-sectional perspective view of a lubrication device 1300 including a lubricant inlet 1342 and lubricant outlet 1344. The lubrication device 1300 may have a lumen 1330 configured to receive a medical device 1390, as shown in FIG. 13. The lubricant inlet 1342 may be configured to receive lubricant and direct the lubricant toward the medical device 1390. The lubricant inlet may receive lubricant from an external lubricant source. In some variations, the lubricant inlet 1342 may be coupled to a positive-pressure source to improve flow of the lubricant to the lubricant outlet 1344 and/or application of the lubricant to the medical device 1390. The lubricant outlet 1344 may be configured to receive excess lubricant and direct it away from the medical device 1390. In some variations, the lubricant outlet may expel excess lubricant from the lubrication device 1300. In some variations, the lubricant outlet 1344 may be coupled to a negative pressure source to improve flow of the lubricant from the lubricant inlet 1342 to the lubricant outlet 1344 or otherwise assist in removal of lubricant from the fluid outlet 1344.

In some variations, a groove 1316 may fluidly couple the lubricant inlet 1342 to the lubricant outlet 1344. The groove 1316 may be configured to apply lubricant to the medical device 1390 as lubricant flows through the groove. For example, the groove 1316 may coil around the medical device 1390 to evenly apply lubricant to the surface of the medical device 1390 as the medical device advances through the first portion 1310 toward the patient. In some variations, the groove 1316 may be in a side wall of the lumen 1330. For example, the groove may be helical and may encircle the medical device 1390 when the medical device 1390 is positioned within the lumen 1330, as shown in FIG. 13.

In some variations, the second portion of the device may comprise one or more waste outlets. The second portion may be configured to remove waste (e.g., residual lubricant) from a medical device and expel the waste from the lubrication device through the one or more waste outlets. For example, waste may be removed from the lubrication device while the waste is being removed from the medical device during retraction of the medical device from a patient. For example, FIG. 13 shows a cross-sectional perspective view of a lubrication device 1300 including one or more waste outlets 1362. A waste outlet 1362 may be in fluid communication with one or more collection chambers 1354 and may remove waste from therein. In some variations, the one or more waste outlets 1362 may be coupled to a negative pressure source to suction waste from the collection chamber 1354.

In some variations, as mentioned previously herein, the second portion may comprise a plurality of seals for removing waste from a medical device. In some of these variations, the seals may be arranged in series along the lumen of the second portion of the device. FIG. 13 shows a lubrication device comprising a plurality of seals 1352 including a first seal and a second seal. The first seal may be configured to remove a first type of waste and second seal may be configured to remove a second different type of waste, or both seals may be configured to remove the same type of waste. For example, the first seal (e.g., distal most seal) may remove some waste from the medical device and a second seal proximal to the first seal may remove additional waste not removed by the first seal. In some variations, one or more seals of the plurality of seal may direct waste into a respective waste collection chamber corresponding to that seal. Additionally or alternatively, one or more seals of the plurality of seals may not be associated with a waste collection chamber.

In some variations, the first portion of the lubrication device may comprise a plurality of bristles to assist in application of lubricant to the medical device. FIG. 14 shows a cross-sectional side view of an exemplary lubrication device 1400 including one or more bristles 1417 to apply lubricant. The first portion of the device 1410 may comprise an application cavity 1413 for applying lubricant to a medical device 1490. The plurality of bristles 1417 may be within the application cavity 1413. For example, one or more bristles of the plurality of bristles 1417 (e.g., each of the plurality of bristles) may be coupled to a side wall of the application cavity and may extend radially inward. In some variations, one or more bristles of the plurality of bristles 1417 may extend into a lumen 1430 of the lubrication device configured to receive a medical device 1490 therein. The application cavity 1413 may be configured to be filled or partially filled with lubricant. Lubricant may be introduced to the application cavity via a lubricant inlet 1442. The plurality of bristles 1417 may be configured to apply lubricant in the application cavity 1413 to the medical device 1490. For example, the plurality of bristles 1417 may be configured to apply the lubricant as the medical device 1490 is advanced through the first portion 1410 of the lubrication device.

In some variations, a second portion of the lubrication device for removing waste may be releasably coupled to a first portion of the lubrication device for applying lubricant. For example, the first portion 1410 of the lubrication device 1400 of FIG. 14 may be releasably coupled to the second portion 1450 of the device 1400. In some variations, the lubrication device 1400 may comprise an actuator 1436 (e.g., button, latch, slide) configured to release the second portion 1450 from the first portion 1410. In some variations, the second portion 1450 may comprise a handle 1438 for manipulating the second portion 1450 when detached from the first portion 1410.

In some variations, the lubrication device may wash a medical device to remove waste. For example, the lubrication device may be configured to wash the medical device using a fluid (e.g., water) to further clean the medical device. For example, as shown in FIG. 14, the lubrication device 1400 may comprise a cleaning chamber 1482. The lubrication device 1400 may comprise a wash inlet 1484 and waste outlet 1486 in fluid communication with the cleaning chamber 1482. A fluid, such as water, may wash the medical device 1490 in the cleaning chamber 1482. In this manner, the fluid may flush waste from the medical device. In some variations, the cleaning chamber 1482 may be fluid-tight. The lubrication device 1400 may comprise one or more fluid-tight seals 1488 to seal the cleaning chamber 1482. The cleaning chamber 1482 may remove waste not removed by the one or more seals 1452. In some variations, the cleaning chamber 1482 may clean the medical device 1490 before the medical device is handled by an operator or a surgical system.

In some variations, the lubrication device may apply lubricant and/or remove waste between one or more seals. For example, in some variations, the lubrication device may apply lubricant between two adjacent seals as a medical device is advanced though the seals. Similarly, the lubrication device may remove waste as the medical device is retracted through two adjacent seals. In some variations, the lubrication device may be configured to couple to a positive pressure source to apply lubricant between adjacent seals, and/or couple to a negative pressure source to remove waste from a portion of the medical device between adjacent seals.

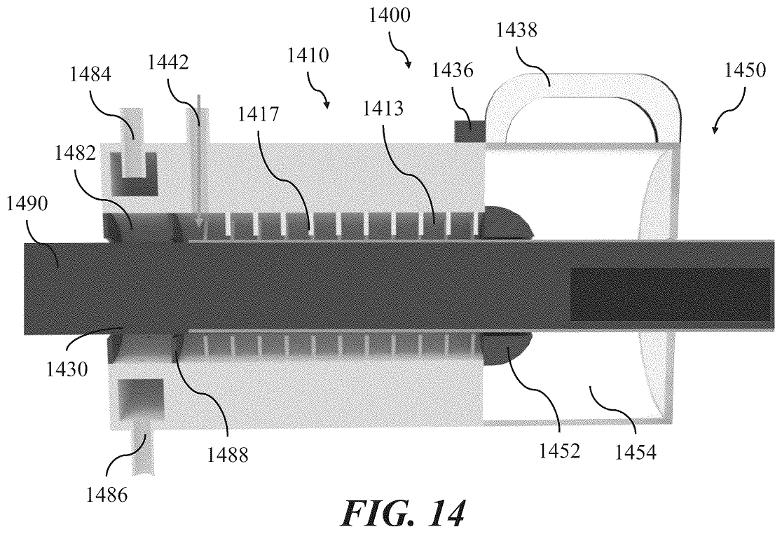

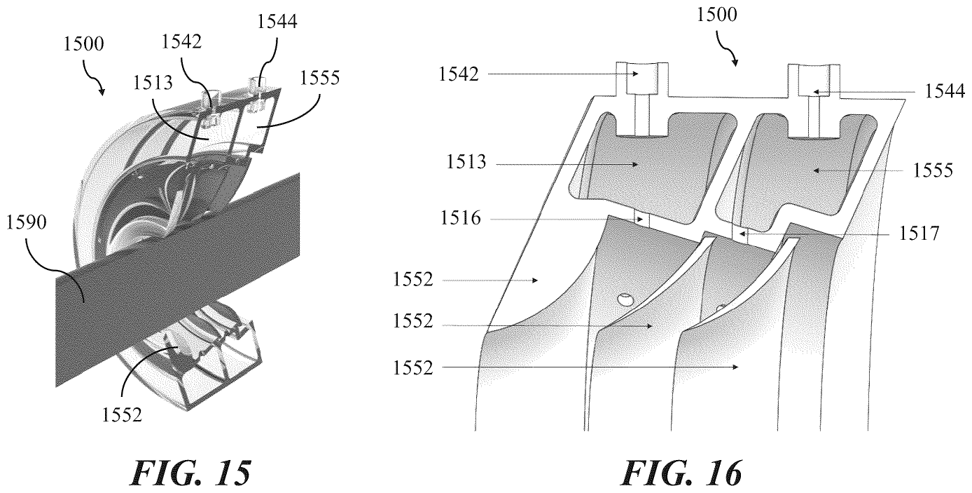

FIGS. 15 and 16 show a cross-sectional perspective view and a cross-sectional side view, respectively, of an exemplary lubrication device 1500 comprising a plurality of seals 1152 where the lubrication device 1500 may be configured to apply lubricant and/or remove waste between the seals. The lubrication device may comprise a lubrication inlet 1542 configured to introduce lubricant to the lubrication device 1500. In some variations, the lubricant inlet 1542 may be configured to couple to a positive pressure source. Optionally, the lubricant inlet 1542 may be threaded so a positive pressure source may be screwed to the inlet 1542. The lubricant inlet 1542 may be in fluid communication with a lubrication cavity 1513. The lubrication cavity 1513 may be positioned radially inward from the lubrication inlet 1542. The lubrication device 1500 may comprise one or more lubricant transfer ports 1516 to allow lubricant to flow from the lubrication cavity 1513 to the plurality of seals 1552. In particular, the one or more lubrication transfer ports 1516 may allow the lubricant to flow into an interstitial space between two adjacent seals 1552. In some variations, the lubrication cavity 1513 may be pressurized by a positive pressure source coupled to the lubrication inlet 1542 to expel lubricant from the one or more transfer ports 1516. In some variations, as described herein, one or more of the seals 1552 may distribute the lubricant onto the medical device 1590.

The lubrication device 1500 may comprise a waste outlet 1544 to remove waste from the lubrication device. The waste outlet 1544 may couple to a negative pressure source to suction waste away from the medical device 1590. Optionally, the waste outlet 1544 may be threaded so a negative pressure source may be screwed to the outlet 1544. The waste outlet may be in fluid communication with a suction cavity 1555. The suction cavity 1555 may be positioned radially inward of the waste outlet 1554. The lubrication device 1500 may further comprise one or more suction inlets 1517. The one or more suction inlets 1517 may be configured to suction waste removed by the seals 1552 away from the medical device 1590. In some variations, one or more suction inlets may be between adjacent seals. The one or more suction inlets 1517 may pull material radially outward and away from the medical device 1590, through the suction cavity 1555, and out of the lubrication device through the waste outlet 1544.

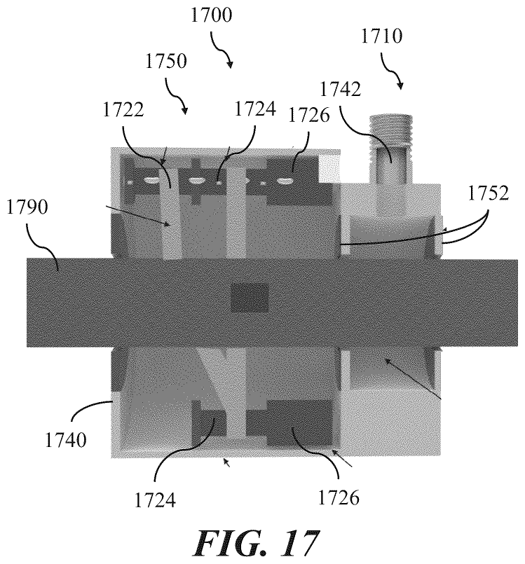

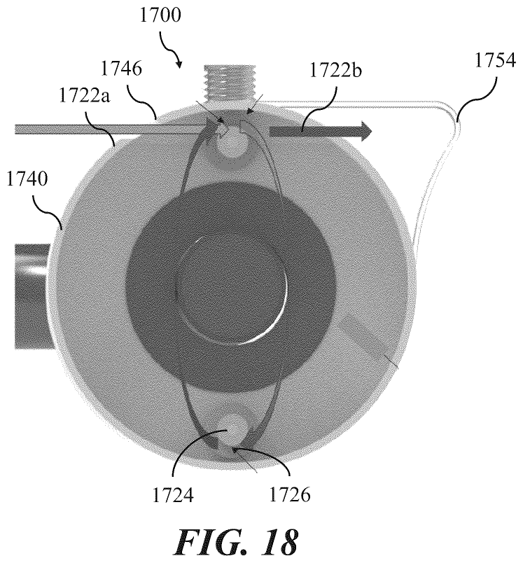

In some variations, a lubrication device may comprise one or more motors to motorize application or removal of the lubricant. In some variations, the lubrication device may comprise a belt configured to wipe waste from a medical device. For example, the belt may be coupled to one or more motors and/or rollers to facilitate wiping the used medical device with the belt. FIGS. 17 and 18 show a cross-sectional side view and a cross-sectional front view, respectively, of an exemplary motorized lubrication device 1700. The lubrication device 1700 may comprise a first portion 1710 configured to apply lubricant to the medical device 1790. For example, the first portion of the lubrication device 1710 may comprise a lubricant inlet 1742 and one or more seals 1752 configured to distribute the lubricant, for example, in an even coating, on a medical device 1790. The lubrication device 1700 may further comprise a second portion 1750 configured to remove waste from the medical device 1790. The second portion 1750 may comprise a belt 1722 to wipe waste from the medical device 1790, one or more pullies 1724 to move the belt, and one or more motors 1726 to actuate the one or more pullies 1724. In some variations, rotation of one or more of the pullies 1724 may translate the belt 1722 relative to the medical device 1790 thereby wiping waste from the medical device. In some variations, the one or more pullies 1724 may position the belt 1722 in contact with the medical device 1790. In some variations, the one or more motors 1726 may drive the one or more pullies 1724 to move the belt 1722. For example, in some variations, one motor may drive two pullies. One or more pullies may not be coupled a motor but may rotate with the belt 1722. The belt may be made from any material suitable to remove waste from the medical device without otherwise damaging the medical device, such as, for example, cloth.

In some variations, the lubrication device may be configured to draw in a clean (e.g., unused) portion of the belt into the housing 1740 and output a contaminated (e.g., used) portion of the belt from the housing for disposal. As shown in FIG. 18, a housing 1740 of the lubrication device 1700 may have a belt inlet 1746 (e.g., slot or other opening in the housing wall) configured to receive a clean portion of the belt 1722a. In some variations, the one or more motorized pullies 1724 may dawn the clean portion of the belt 1722a into the lubrication device 1700 (e.g., into the housing 1740). The lubrication device 1700 may comprise a disposal chamber 1754, for example, aligned with the belt inlet 1746 and extending radially outward from the housing, to collect a contaminated portion of the belt 1722b. In some variations, the one or more motorized pullies 1724 may feed the contaminated portion of the belt 1722b into the disposal chamber 1754.

### II. Methods

Also described herein are methods for lubricating a medical device. For example, a method of lubricating a medical device may generally include applying lubricant to the medical device using a lubrication device as the medical device is advanced through a lumen of the lubrication device, removing lubricant from the medical device using the lubrication device as the medical device is retracted through the lubrication device, and capturing the lubricant removed from the medical device within the lubrication device.

In some variations, lubricant may be pumped into the lubrication device. Lubricant may be introduced to a first portion of the lubrication device to be applied to a medical device. Lubricant may be pumped into the lubrication device prior to applying lubricant to the medical device. In some variations, lubricant may be pumped into the lubrication device prior to the lubrication device receiving the medical device for lubrication. In some variations, lubricant may be pumped into the lubrication device while the medical device is being advanced through the lubrication device.

In some variations, a method of applying lubrication to a medical device using a lubrication device may comprising removing one or more plugs or covers from the lubrication device. In some variations, a plug and/or cover may be removed prior to the lubrication device receiving the medical device. In other variations, the plug and/or cover may be removed after the lubrication device has received the medical device. In some variations, a method of applying lubrication using a lubrication device may comprise pressurizing lubricant within the device. For example, a reservoir holding lubricant in the lubrication device may be pressurized, as described herein.

In some variations, a method may comprise applying lubricant to a medical device. Lubricant may be applied to the medical device using a lubrication device as described herein. In some variations, lubricant may be applied as the medical device is advanced towards a patient and/or as the medical device is advanced through the lubrication device. For example, lubricant may be applied while the medical device moves in a distal direction. In some variations, lubricant may be applied within the lubrication device. For example, the medical device may be lubricated using a first portion of a lubrication device. The medical device may be lubricated within a lumen of the lubrication device. In some variations, applying lubricant may include transferring lubricant from a pressurized reservoir to the medical device.

In some variations, applying lubricant to the medical device may comprise flowing lubricant through the lubrication device. In some variations, applying lubricant to the medical device may comprise applying lubricant around an outer surface of the medical device using a helical application. For example, applying lubricant may coil the lubricant around the medical device. In some variations, applying lubricant to the medical device may include using one or more bristles to apply the lubricant. In some variations, a coating of lubricant applied to the medical device may be between about 0.1 mm and about 1 mm, including all subranges and values therebetween.

In some variations, a method may further include evenly distributing the lubricant after applying the lubricant to the medical device. In some variations, one or more seals of the lubrication device may be used to more evenly apply the lubricant. In some variations, methods may further include expelling or otherwise transferring excess lubricant from the lubrication device to an external waste storage, such a medical waste container. For example, waste including excess lubricant may be disposed of by detaching a waste collection chamber from the lubrication device and moving the collection chamber the external waste storage.

In some variations, a method may comprise removing lubricant and/or other waste from the medical device. Waste may be removed from the medical device using the lubrication devices themselves as described herein. In some variations, waste may be removed from the medical device as the medical device is retracted from a patient and/or through the lubrication device proximally. For example, waste may be removed from the medical device as the medical device moves through a second portion of the lubrication device. For example, waste may be removed from the medical device as the medical device translates relative to the second portion of the lubrication device.

In some variations, removing waste (e.g., lubricant, bodily fluids, or other contaminants) from the medical device may comprise scraping waste from the medical device. In some variations, waste may be scraped off using one or more seals of the lubrication device. In some variations, removing the waste may prevent waste from reaching one or more other portions of the lubrication device, an operator, or other components of a surgical system (e.g., control device). In some variations, methods may further include applying negative pressure to remove excess lubricant. For example, negative pressure may be applied to the medical device and/or waste while either is within a lubrication device. In some variations, removing waste may include washing the medical device with a fluid to remove waste from the device and/or wiping waste off the medical device (e.g., using a seal or other component of the lubrication device). For example, waste may be removed by wiping the medical device with a belt.

In some variations, the method may further comprise capturing the waste removed from the medical device. The removed waste may be collected using a lubrication device as described herein. For example, removed waste may be collected using a collection chamber of a lubrication device. In some variations, capturing the waste may include directing a flow of the removed waste into a collection chamber. For example, the flow of the removed waste may be directed using a seal of the lubricate device. In some variations, the method may comprise disposing of the collected waste. In some variations, the waste may be disposed of by disposing of a portion of the lubrication device containing the waste.

The acts performed as part of the methods herein may be ordered in any suitable way. Accordingly, various methods may be constructed in which acts are performed in an order different than illustrated, which may include performing some acts simultaneously, even though shown as sequential acts in illustrative examples.

While certain variations are described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive variations described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain using no more than routine experimentation, many equivalents to the specific inventive variations described herein. It is, therefore, to be understood that the foregoing variations are presented by way of example only and that, within the scope of the appended claims and equivalents thereto; inventive variations may be practiced otherwise than as specifically described and claimed. Inventive variations of the present disclosure are directed to each individual feature and/or method described herein. In addition, any combination of two or more such features and/or methods, if such features and/or methods are not mutually inconsistent, is included within the inventive scope of the present disclosure.

1. A lubrication device, comprising:
a first portion configured to apply a lubricant to an outer surface of an elongate body of a medical device; and
a second portion distal to the first portion, the second portion comprising a seal configured to remove the lubricant from the elongate body and a waste collection chamber configured to receive and contain the removed lubricant.

2. The lubrication device of claim 1, wherein the first and second portions comprise a first and a second lumen therethrough respectively, wherein the first and second lumens are configured to receive the elongate body of the medical device.

3. The lubrication device of claim 2, wherein the first portion comprises a lubricant reservoir.

4. The lubrication device of claim 3, wherein the lubricant reservoir is pressurized by one or more biasing members.

5. The lubrication device of claim 3, wherein the lubricant reservoir is pressurized by a pressurized gas.

6. The lubrication device of claim 3, wherein the lubricant reservoir concentrically surrounding the first lumen.

7. The lubrication device of claim 3, wherein the first portion further comprises a plurality of lubricant transfer ports fluidly coupling the lubricant reservoir and the first lumen.

8. The lubrication device of claim 2, wherein the seal is configured to contact the outer surface of the elongate body of the medical device received within the second lumen.

9. The lubrication device of claim 1, wherein the seal is configured to distribute the lubricant applied by the first portion when the medical device is advanced through the lubrication device in a distal direction.

10. The lubrication device of claim 1, wherein the seal is configured to remove lubricant from the outer surface of the elongate body of the medical device when the medical device is retracted through the lubrication device in a proximal direction.

11. The lubrication device of claim 1, wherein the seal is configured to direct the removed lubricant into the waste collection chamber.

12. The lubrication device of claim 1, wherein the seal is biased radially inward.

13. The lubrication device of claim 1, wherein the second portion is releasably coupled to the first portion.

14. The lubrication device of claim 1, wherein the first portion comprises a lubricant inlet configured to receive the lubricant from an external source.

15. The lubrication device of claim 1, wherein the second portion further comprises a lubricant outlet.

16. The lubrication device of claim 15, wherein the lubricant outlet is configured coupled to a negative pressure source.

17. A method of lubricating a medical device, comprising:
applying lubricant to an outer surface of an elongate body of the medical device as the elongate body is advanced through a lumen of a lubrication device;
removing lubricant from the elongate body using the lubrication device as the elongate body is retracted through the lubrication device; and
capturing the removed lubricant within the lubrication device.

18. The method of claim 17, wherein the removed lubricant is captured in a waste collection chamber of the lubrication device.

19.-20. (canceled)

21. A surgical system, comprising:
a medical device;
a drive device releasably coupled to the medical device, the drive device configured to control the medical device; and
a lubrication device slidably coupled to the medical device distal to the drive device, wherein the lubrication device comprises:
a first portion configured to apply a lubricant to an outer surface of the medical device; and
a second portion distal to the first portion, the second portion comprising a seal configured to remove the lubricant from the medical device and a waste collection chamber configured to receive and contain the removed lubricant.

22. The system of claim 21, wherein the seal is biased radially inward.
